(19) United States
(12) Patent Application Publication
(10) Pub. No.: US 2026/0166126 A1
(43) Pub. Date: Jun. 18, 2026

(54) COMPOSITIONS AND METHODS OF USING NITRATION RESISTANT CCL2 CHEMOKINES

(71) Applicant: BOARD OF REGENTS, THE UNIVERSITY OF TEXAS SYSTEM, Austin, TX (US)

(72) Inventors: Neelam MUKHERJEE, Austin, TX (US); Robert SVATEK, Austin, TX (US); Shaun OLSEN, Austin, TX (US)

(21) Appl. No.: 19/129,474

(22) PCT Filed: Nov. 14, 2023
(86) PCT No.: PCT/US2023/079616

§ 371 Date: May 13, 2025

Related U.S. Application Data
(60) Provisional application No. 63/425,128, filed on Nov. 14, 2022.

(51) Int. Cl. A61K 38/19; A61P 35/00; C07K 14/52

(52) U.S. Cl. CPC A61K 38/195; A61P 35/00; C07K 14/521

(57) ABSTRACT

The present disclosure relates to compositions comprising nitration resistant CCL2 recombinant proteins and methods of use thereof.

### CROSS REFERENCE TO RELATED APPLICATIONS

This PCT application claims priority to, and the benefit of, U.S. Provisional Patent Application No. 63/425,128, filed Nov. 14, 2022, entitled “COMPOSITIONS AND METHODS OF USING NITRATION RESISTANT CCL2 CHEMOKINES,” which is incorporated by reference herein in its entirety.

### STATEMENT REGARDING FEDERALLY SPONSORED RESEARCH

This invention was made with Government Support under Grant No. CA178204 awarded by the National Institutes of Health. The Government has certain right in the invention.

### REFERENCE TO SEQUENCE LISTING

The sequence listing submitted on Nov. 14, 2023, as an .XML file entitled “11024-013WO1_ST26.xml” created on Nov. 13, 2023, and having a file size of 111,598 bytes is hereby incorporated by reference pursuant to 37 C.F.R. § 1.52(e)(5).

### FIELD

The present disclosure relates to compositions comprising nitration resistant CCL2 recombinant proteins and methods of use thereof.

### BACKGROUND

Immune cells are an important component of the body's natural defense against tumor growth. Immune therapy has revolutionized the approach to managing cancers in various tissues. However, complete response is seen only in a small percentage of patients. In addition, a limitation in improving immune therapy is lack of understanding of how immune cells are recruited to the tumor environment. Chemokines are small molecules the direct immune cells to their target destinations. In cancer, they become particularly important for targeting tumor-fighting immune cells to specific sites. There is a need to develop a chemokine-based immune therapy to treat cancer.

Thus, the present disclosure addresses the aforementioned problems and other shortcomings associated with traditional immune therapies.

### SUMMARY

The present disclosure provides compositions and expression vectors comprising a nitration resistant C—C motif ligand 2 (CCL2) chemokine. The present disclosure also provides methods of treating, preventing, decreasing, reducing, and/or ameliorating cancer.

In one aspect, disclosed herein is a composition comprising a nitration resistant C—C motif ligand 2 (CCL2) chemokine and a pharmaceutically acceptable salt, carrier, excipient, diluent, or surfactant, wherein the nitration resistant CCL2 comprises one or more mutations of a tyrosine (Tyr), a tryptophan (Trp), a methionine (Met), or a cysteine (Cys) amino acid.

In some embodiments, the nitration resistant CCL2 chemokine comprises the one or more mutation at Tyr18, Tyr33, or Trp64 of SEQ ID NO: 1 or 18. In some embodiments, the nitration resistant CCL2 chemokine comprises the one or more mutation at Tyr36, Tyr51, or Trp82 of SEQ ID NO: 9 or 26. In some embodiments, the nitration resistant CCL2 chemokine comprises at least 70% sequence identity to any one of SEQ ID NO: 2-8. SEQ ID NO: 19-25, or variants thereof. In some embodiments, the nitration resistant CCL2 chemokine comprises at least 80% k sequence identity to any one of SEQ ID NO: 2-8, SEQ ID NO: 19-25, or variants thereof. In some embodiments, the nitration resistant CCL2 chemokine comprises at least 90% sequence identity to any one of SEQ ID NO: 2-8, SEQ ID NO: 19-25, or variants thereof. In some embodiments, the nitration resistant CCL2 chemokine comprises any one of SEQ ID NO: 2-8, SEQ ID NO: 19-25, or variants thereof.

In some embodiments, the Tyr is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Trp is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Met is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Cys is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine.

In one aspect, disclosed herein is an expression vector comprising a nucleic acid sequence encoding a nitration resistant C—C motif ligand 2 (CCL2) chemokine, wherein the nucleic acid sequence encodes one or more mutation of a tyrosine (Tyr), a tryptophan (Trp), a methionine (Met), or a cysteine (Cys) amino acid. In some embodiments, the nucleic acid sequence comprises at least 70% sequence identity to any one of SEQ ID NO: 11-17. SEQ ID NO: 28-34, or variants thereof. In some embodiments, the nucleic acid sequence comprises at least 80% sequence identity to any one of SEQ ID NO: 11-17, SEQ ID NO: 28-34, or variants thereof. In some embodiments, the nucleic acid sequence comprises at least 90% sequence identity to any one of SEQ ID NO: 11-17, SEQ ID NO: 28-34, or variants thereof. In some embodiments, the nucleic acid sequence comprises any one of SEQ ID NO: 11-17, SEQ ID NO: 28-34, or variants thereof.

In one aspect, disclosed herein is a method of treating or preventing a cancer in a subject, the method comprising administering a pharmaceutically effective amount of a composition comprising a nitration resistant C—C motif ligand 2 (CCL2) chemokine and a pharmaceutically acceptable carrier comprising a salt, carrier, excipient, diluent, surfactant, buffer, or nanoparticle, wherein the nitration resistant CCL2 comprises one or more mutations of a tyrosine (Tyr), a tryptophan (Trp), a methionine (Met), or a cysteine (Cys) amino acid.

In some embodiments, the nitration resistant CCL2 chemokine comprises at least 70% sequence identity to any one of SEQ ID NO: 2-8. SEQ ID NO: 19-25, or variants thereof. In some embodiments, the nitration resistant CCL2 chemokine comprises at least 80% sequence identity to any one of SEQ ID NO: 2-8, SEQ ID NO: 19-25, or variants thereof. In some embodiments, the nitration resistant CCL2 chemokine comprises at least 90% sequence identity to any one of SEQ ID NO: 2-8, SEQ ID NO: 19-25, or variants thereof. In some embodiments, the nitration resistant CCL2 chemokine comprises any one of SEQ ID NO: 2-8, SEQ ID NO: 19-25, or variants thereof.

In some embodiments, the nitration resistant CCL2 cannot be nitrated at a tyrosine (Tyr), a tryptophan (Trp), a methionine (Met), or a cysteine (Cys) amino acid. In some embodiments, the nitration resistant CCL2 cannot be nitrated at Tyr18, Tyr33, or Trp64 of SEQ ID NO: 1 or 18. In some embodiments, the nitration resistant CCL2 cannot be nitrated at Tyr36, Tyr51, or Trp82 of SEQ ID NO: 35 or 36.

In some embodiments, the nitration resistant CCL2 outcompetes a native CCL2. In some embodiments, the nitration resistant CCL2 recruits a CD8⁺ T cell or a monocytic myeloid cell. In some embodiments, the method prevents nitration of the nitration resistant CCL2. In some embodiments, the nitration resistant CCL2 comprises an anti-tumor chemokine. In some embodiments, nitration of the CCL2 is reduced or eliminated by 10% or more compared to a control.

In some embodiments, the nitration resistant CCL2 is administered by an injection or by an instillation. In some embodiments, the cancer is a bladder cancer, a breast cancer, a colon cancer, a glioblastoma, or a prostate cancer. In some embodiment, the subject is a human or a rodent.

### BRIEF DESCRIPTION OF FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows the effect nitration resistant recombinant CCL2 (rCCL2^{NR}) on tumor growth.

FIGS. 2A, 2B, 2C, 2D, and 2E show that CCL2 is increased during carcinogen exposure but protects bladder from tumor development and growth. FIG. 2A shows B6 WT mice were challenged with MB49 tumors and sacrificed on day 21 and immune profiling by flow cytometry was carried out. FIG. 2B shows that B6 mice were fed water +/−0.05% BBN. After 4 months, urine was collected and CCL2 was measured with ELISA. FIG. 2C shows that wildtype (WT) and CCL2^{KO} male mice were given BBN in drinking water for 4.5 months to induce BC, and then sacrificed for histopathologic examination of bladders. FIGS. 2D and 2E show the female or male (FIG. 2D) WT vs CCL2^{KO} and (FIG. 2E) WT vs CCR2KO mice were challenged with orthotopic 80,000 MB49 cells and sacrificed at day 17 for determination of bladder weights. P-fisher's exact or two-tailed, unpaired t-test or two-tails Mann-Whitney.

FIGS. 3A, 3B, 3C, and 3D show the effects of CCL2 inhibition is environment dependent. FIGS. 3A, 3B, 3C, and 3D show that female WT mice were challenged in the mammary fat pads or skin and bladder with AT-3 mammary tumors or MB49 bladder tumors and treated with or without CCL2 neutralizing antibody (αCCL2) and followed for growth or bladder weights were measured. Difference in growth compared with 2-way ANOVA, unpaired t-test *P<0.05, **P<0.01, ***P<0.001.

FIGS. 4A, 4B, 4C, 4D, 4E, 4F, and 4G show that CCR2 protects BC growth by recruiting T cells. B6 WT and CCL2^{KO} mice were challenged with MB49 tumors and sacrificed on day 21. FIG. 4A shows that bladders examined for T cells and NK cells (by flow, plotted as % of CD45⁺ cell). FIG. 4B shows that mice were given α-CD3, or α-AsialoGM1. FIG. 4C shows that mice were given α-CD4 or α-CD8. *P<0.05 unpaired two-tailed t-test. FIG. 4D shows that B6 WT and Rag1^{KO} mice were challenged with MB49 bladder tumors and treated with or without CCL2 neutralizing antibody (αCCL2) and bladder weights were measured. FIG. 4E shows that WT and CCR2KO female mice were challenged with MB49 tumors and sacrificed on day 21 and bladder weights were measured. FIG. 4F shows CCR2^{KO} female mice were adoptively transferred with T cells from either B6 WT mice and CCR2^{KO} mice and survival was followed. FIG. 4G shows that irradiated TCRα^{KO} mice were reconstituted with 1:1 mixtures of bone marrows from CCR2⁺ (CD45.1) and CCR2^{-/-} (CD45.2) mice and challenged orthotopically with MB49 cells. Frequencies of CCR2⁺ vs CCR2⁻ bladder tumor infiltrating T cells are indicated.

FIGS. 5A, 5B, and 5C show that recombinant CCL2 decreases bladder tumor growth. B6 female mice were challenged orthotopically with 80,000 MB49 bladder cancer cells and were divided into four groups. MB49 orthotopic tumors are treated with PBS, Gemcitabine, recombinant CCL2 (rCCL2), and Gemcitabine and rCCL2. Treatment was given intravesically through a transurethral catheter and instilled for 1 hour weekly. FIGS. 5A, 5B, and 5C show that survival was monitored over a period of 2 months in WT mice, TCRα knockout mice, or CCR2 knockout mice. Difference in survival curves were compared with a two-sided log-rank test.

FIGS. 6A, 6B, 6C, and 6D show that nitration of CCL2 reduces the therapeutic efficacy of recombinant CCL2. FIG. 6A shows that MB49 bladder tumors were transplanted orthotopically into female mice and subsequently analyzed. Confocal microscopy showing CCL2 (red) is nitrated (yellow) in bladder tumors (blue-nucleus and green-CK20 epithelium). FIG. 6B shows that colocalization is measured by calculation of Mander's colocalization coefficient. FIG. 6C shows that WT BL6 female mice were challenged intravesically with MB49. Five days after tumor challenge, mice were treated weekly (×4) with intravesical rCCL2, n-rCCL2, or PBS control, rCCL2 was nitrated with 1 mM peroxynitrite×10 minutes. Mice were followed for survival, log-rank test. FIG. 6D shows the bladder tumor infiltrating T cells and monocytic myeloid cells were detected by flow cytometry.

FIG. 7 shows the amino acid sequences of human wild-type CCL2 within the pET-NTEV plasmid (SEQ ID NO: 9) and the final product after TEV protease cleavage (SEQ ID NO: 1).

FIG. 8 shows the amino acid sequences of mouse wild-type CCL2 within the pET-NTEV plasmid (SEQ ID NO: 26) and the final product after TEV protease cleavage (SEQ ID NO: 18).

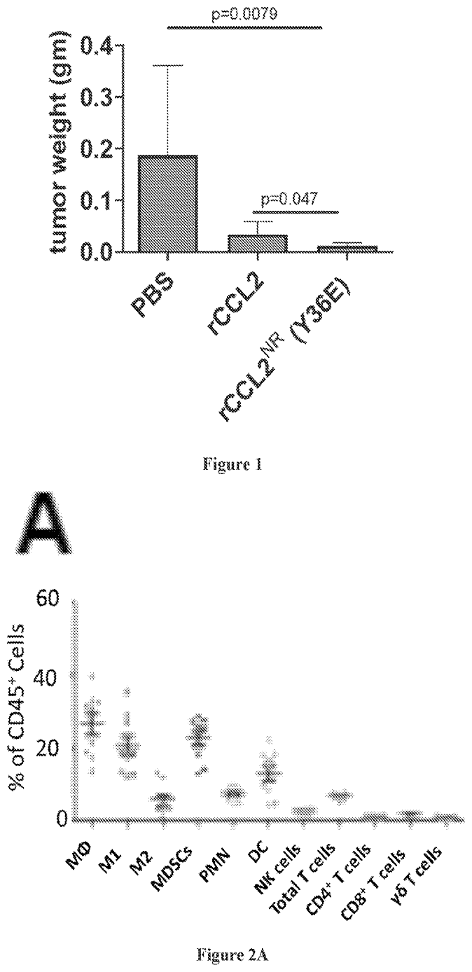

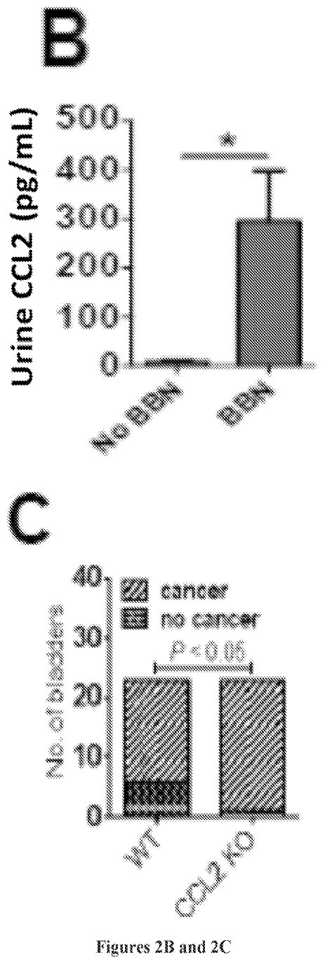

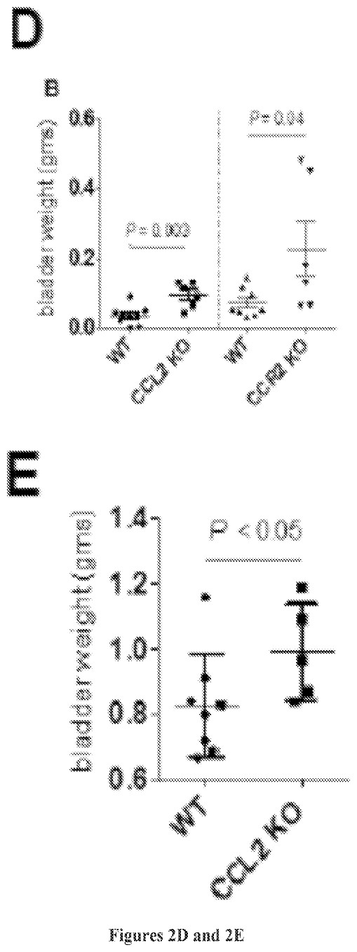

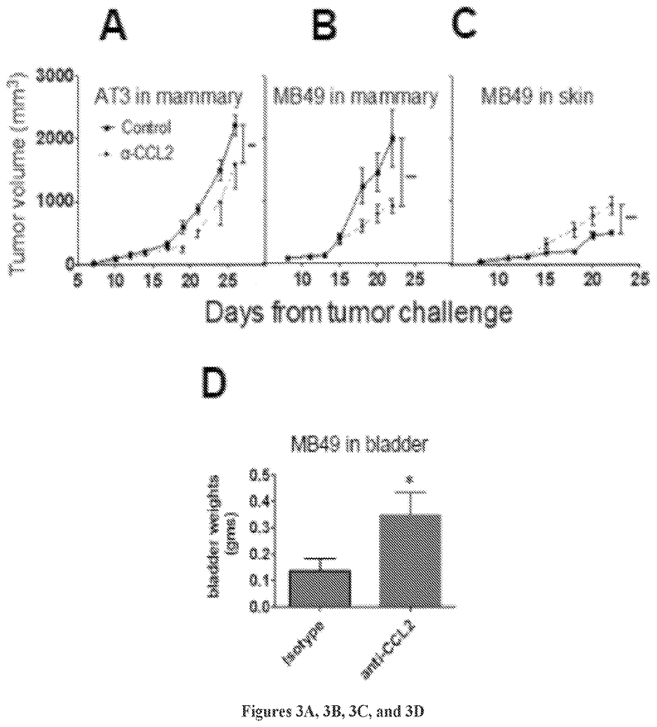

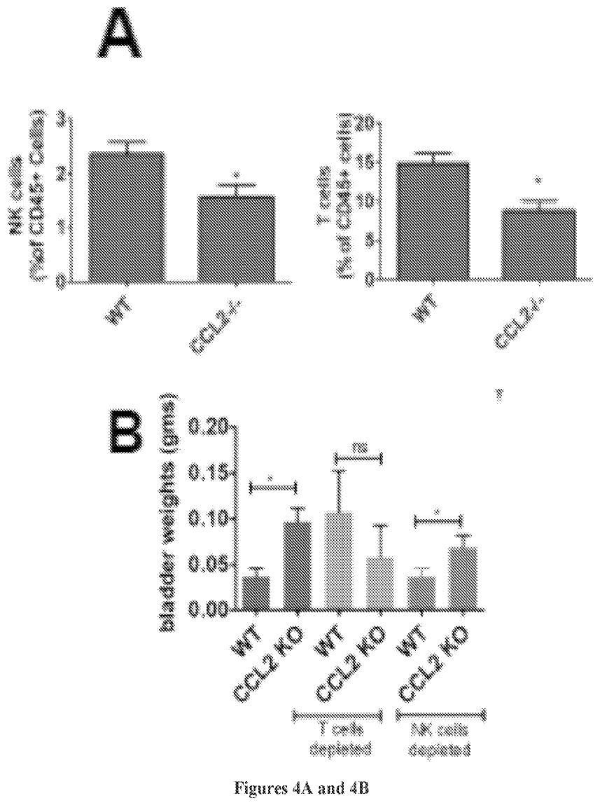

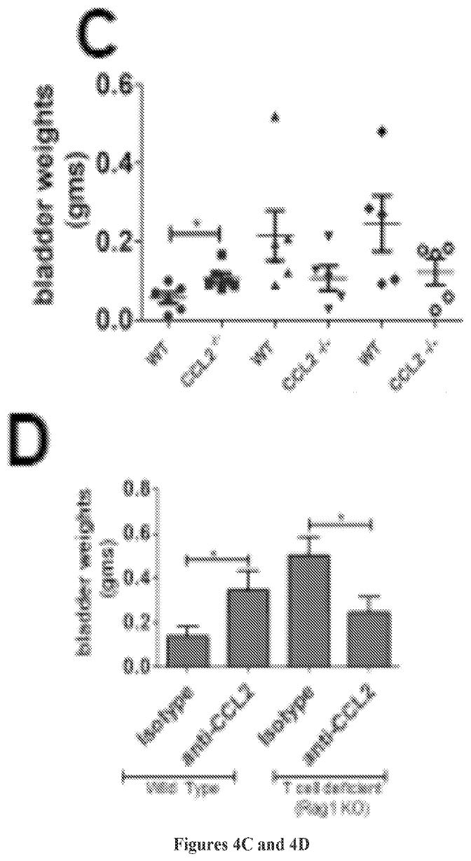

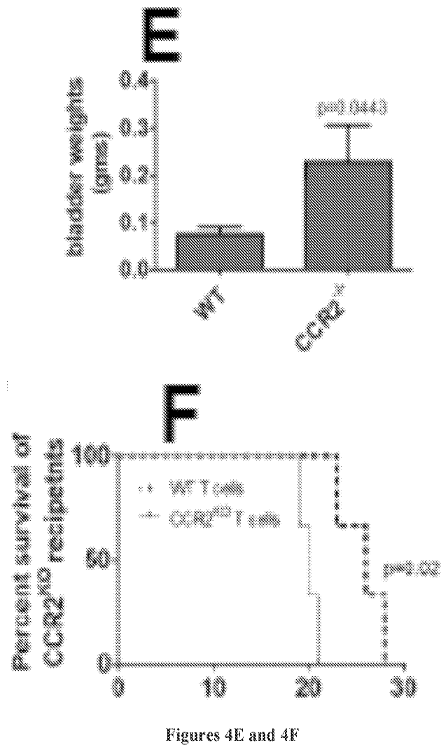

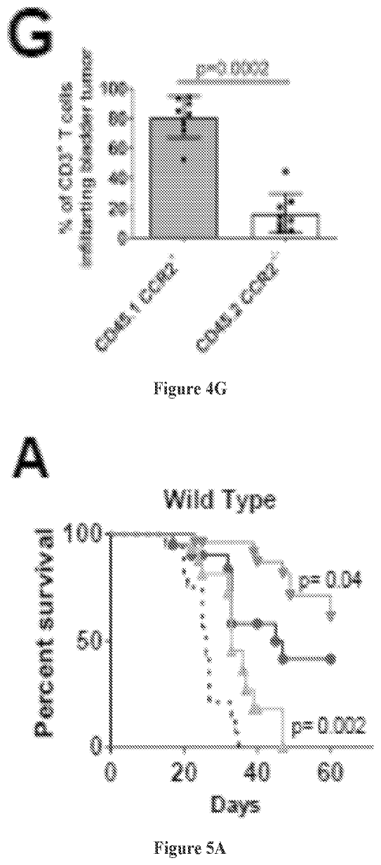

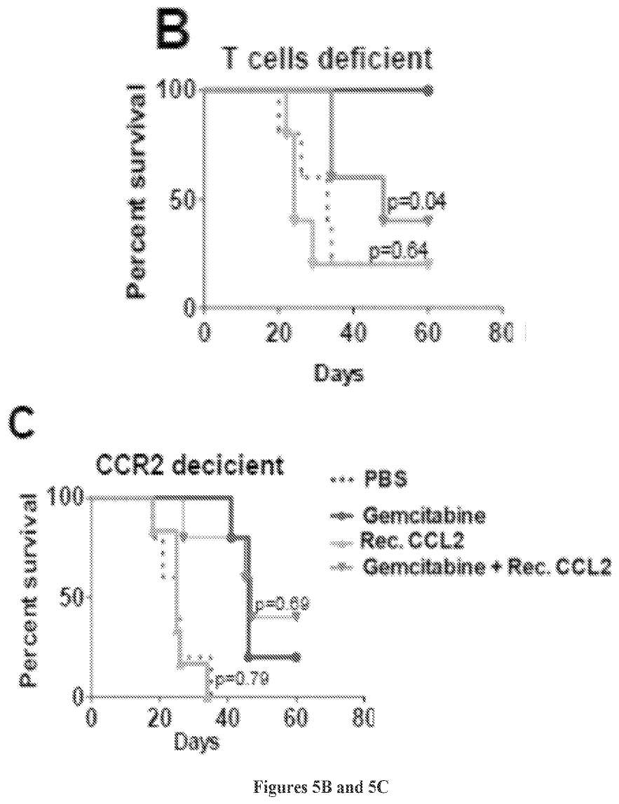

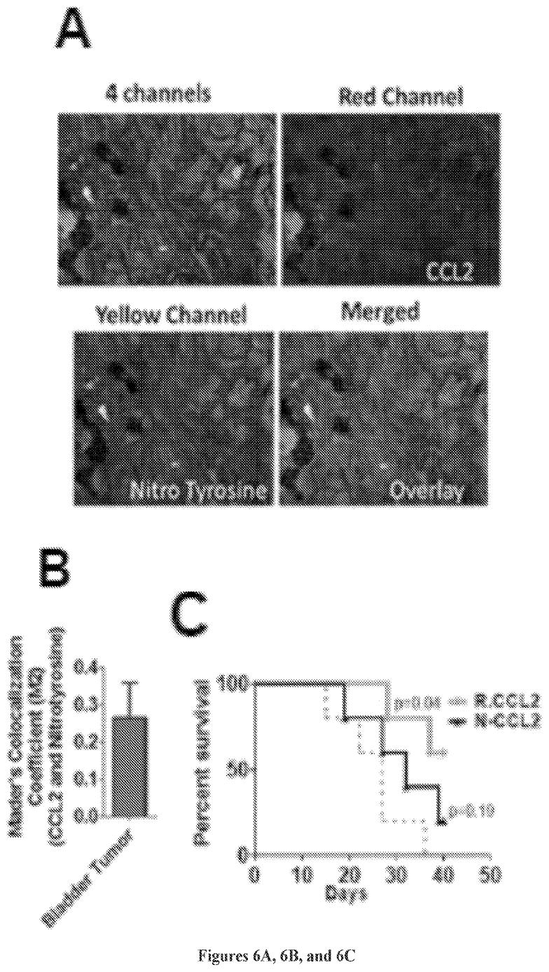

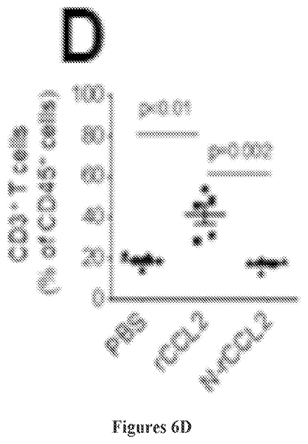

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

The following definitions are provided for the full understanding of terms used in this specification.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally.” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction.” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Inhibitors” of expression or of activity are used to refer to inhibitory molecules, respectively, identified using in vitro and in vivo assays for expression or activity of a described target protein. e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The terms “prevent,” “preventing,” “prevention,” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

A “T cell” refers to a type of lymphocyte that is one of the most important white blood cells of the immune system. T cells can be distinguished from other lymphocytes by the presence of a T-cell receptor (TCR) on their cell surface. The immune-mediated cell death function of T cells is carried by two major subtypes: CD8⁺ “killer” T cells and CD4⁺ “helper T cells.

As used herein, the term “compound,” refers to a chemical substance consisting of two or more different types of atoms or chemical elements in a fixed stoichiometric proportion. These compounds have a unique and defined chemical structure held together in a defined spatial arrangement by chemical bonds. Chemical compounds can be held together by covalent bonds, ionic bonds, metallic ions, or coordinate covalent bonds.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

The terms “treat,” “treating.” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

As used herein, a “solution” refers to a liquid mixture wherein a minor component (solute) is uniformly distributed within a major component (solvent).

The term “detection,” “detect,” or “detecting” refers to an output signal released for the purpose of sensing of physical phenomenon. An event or change in environment is sensed and signal output released in the form of light.

By “eliminating,” or other forms of the word, such as “eliminate.” “eliminated,” or “elimination,” it is referring to remove an occurrence, characteristic, or object from consideration, or to prevent said occurrence, characteristic, or object from occurring or existing.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

A “protein.” “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999. Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to nitration (attachment of a nitro group (—NO₂) to an amino acid including but not limited to tyrosine, tryptophan, cysteine, and methionine residues), carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides). S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda. Md., at its website. The BLAST software suite includes various sequence analysis programs including “blastp.” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit, or more biological activities associated with binding a ligand and/or binding DNA at a specific binding site.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda. Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

### Nitration Resistant Ccl2 Compositions

Chemokines are a large family of low molecular weight proteins that are able to control the migration and residence of various cells, including immune cells. Of the two main groups of chemokines, the C—C chemokines are inflammatory mediators of monocyte and lymphocyte, such as B and T cell, migration into tumor sites. The C—C motif ligand 2 chemokine (CCL2) has been implicated in cancer promotion by the recruitment of immunosuppressive monocytes. However, CCL2 also recruits cytotoxic T cells to tumors, thereby contributing to the antitumor immunity in the tumor microenvironment and providing evidence of CCL2 having a defensive role against cancer.

With the discovery of post-translational nitration, it was shown that nitration, or adding a nitro group (—NO₂) to an amino acid residue, alters protein structures and function as well as is involved in pathophysiological diseases, such as cancer. Nitration generally occurs at tyrosine, tryptophan, cysteine, methionine, and phenylalanine amino acids.

Recombinant CCL2 (rCCL2) is therapeutic anticancer agent, however effects are lost when rCCL2 is nitrated. Other studies assume the CCL2 is always active, but fail to consider that post-translational modifications, such as nitration, affects CCL2 activity. Therefore, there is a need to develop a nitration resistant CCL2 chemokine, or CCL2 nitration inhibitor to prevent inactivation of CCL2 and promote its anticancer activities.

The present disclosure provides compositions and expression vectors comprising a nitration resistant C—C motif ligand 2 (CCL2) chemokine. It should be noted that human or mouse wild-type CCL2 sequences, or fragments thereof, can be modified to reduce CCL2 nitration

In one aspect, disclosed herein is a composition comprising a nitration resistant C—C motif ligand 2 (CCL2) chemokine and a pharmaceutically acceptable salt, carrier, excipient, diluent, or surfactant, wherein the nitration resistant CCL2 comprises one or more mutations of a tyrosine (Tyr), a tryptophan (Trp), a methionine (Met), or a cysteine (Cys) amino acid.

Therefore, in some embodiments, the nitration resistant CCL2 comprises a mutation of a tyrosine amino acid. In some embodiments, the nitration resistant CCL2 comprises a mutation of a tryptophan amino acid. In some embodiments, the nitration resistant CCL2 comprises a mutation of a cysteine amino acid. In some embodiments, the nitration resistant CCL2 comprises a mutation of a methionine amino acid.

Multiple amino acid molecules can be mutated on the same CCL2, so that 2, 3, 4, 5, 6, 7, 8, 9, or 10 or more amino acid molecules can be mutated so that they differ from the naturally occurring amino acid sequence. For example, the mutations can comprise 1, 2, 3, 4, 5, 6, 7, 8, 9, or 10 or more mutations to tyrosine residues, 1, 2, 3, 4, 5, 6, 7, 8, 9, or 10 or more mutations to tryptophan residues, 1, 2, 3, 4, 5, 6, 7, 8, 9, or 10 or more mutations to phenylalanine residues, 1, 2, 3, 4, 5, 6, 7, 8, 9, or 10 or more mutations to methionine residues, and 1, 2, 3, 4, 5, 6, 7, 8, 9, or 10 or more mutations to cysteine residues. It is noted that any combination of these mutations can occur. One of skill in the art can readily determine which of these combinations of mutations will result in a nitration resistant CCL2 molecule.

The present disclosure contemplates the relative locations of mutations on CCL2 variants. In some embodiments, the nitration resistant CCL2 is a recombinant CCL2 polypeptide, or a variant thereof. The compositions herein disclose a CCL2 fragment comprising mutations to Tyr18, Tyr33, and/or Trp64. It should be noted that the mutations to Tyr18, Tyr33, and/or Trp64 of the CCL2 fragment (such as, for example SEQ ID NO: 1 or SEQ ID NO: 18) correspond to Tyr36, Tyr51, and/or Trp 82 of a full-length wild-type CCL2 polypeptide (such as, for example SEQ ID NO: 9, SEQ ID NO: 35, SEQ ID NO: 26, or SEQ ID NO: 36).

Disclosed herein is SEQ ID NO: 1, SEQ ID NO: 9, and SEQ ID NO: 35, which are the amino acid sequence variants that represent wild-type CCL2 in humans.

SEQ ID NO: 9 represents a CCL2 variant incorporated and/or in an expression vector, including, but not limited to a plasmid (such as, for example pET29-NTEV), a viral vector, a virus, and nanoparticle. In some embodiments, SEQ ID NO: 9 comprises one or more mutations to Tyr36, Tyr51, and/or Trp 82. In some embodiments, SEQ ID NO: 9 comprises any number of modifications, including but not limited to modifications at Tyr36 (CCL2 Y36X); modifications to Tyr51 (CCL2 Y51X); and modifications to Trp82 (CCL2 W82X); wherein X is any amino acid excluding tyrosine, tryptophan, cysteine, and methionine.

SEQ ID NO: 1 represents a fragment of SEQ ID NO: 9 after proteolytic cleavage. In some embodiments, SEQ ID NO: 1 comprises one or more mutations to Tyr18, Tyr33, and/or Trp64. In some embodiments, SEQ ID NO: 1 comprises any number of modifications, including but not limited to modifications at Tyr18 (SEQ ID NO: 2-CCL2 Y18X); modifications to Tyr33 (SEQ ID NO: 3-CCL2 Y33X); and modifications to Trp64 (SEQ ID NO: 4-CCL2 W64X); wherein X is any amino acid excluding tyrosine, tryptophan, cysteine, and methionine.

SEQ ID NO: 35 represents a CCL expressed in humans. In some embodiments, SEQ ID NO: 35 comprises one or more mutations to Tyr36. Tyr51, and/or Trp82. In some embodiments, SEQ ID NO: 35 comprises any number of modifications, including but not limited to modifications at Tyr36 (CCL2 Y36X); modifications to Tyr51 (CCL2 Y51X); and modifications to Trp82 (CCL2 W82X); wherein X is any amino acid excluding tyrosine, tryptophan, cysteine, and methionine.

Also disclosed herein is SEQ ID NO: 18, SEQ ID NO: 26, and SEQ ID NO: 36, which are the amino acid sequence variants that represent wild-type CCL2 in mice.

SEQ ID NO: 18 represents a CCL2 variant incorporated and/or in an expression vector, including, but not limited to a plasmid (such as, for example pET29-NTEV), a viral vector, a virus, and nanoparticle. In some embodiments, SEQ ID NO: 18 comprises one or more mutations to Tyr36, Tyr51, and/or Trp 82. In some embodiments, SEQ ID NO: 18 comprises any number of modifications, including but not limited to modifications at Tyr36 (CCL2 Y36X); modifications to Tyr5l (CCL2 Y51X); and modifications to Trp82 (CCL2 W82X); wherein X is any amino acid excluding tyrosine, tryptophan, cysteine, and methionine.

SEQ ID NO: 26 represents a fragment of SEQ ID NO: 18 after proteolytic cleavage. In some embodiments, SEQ ID NO: 26 comprises one or more mutations to Tyr18, Tyr33, and/or Trp64. In some embodiments, SEQ ID NO: 26 comprises any number of modifications, including but not limited to modifications at Tyr18 (SEQ ID NO: 19-CCL2 Y18X); modifications to Tyr33 (SEQ ID NO: 20-CCL2 Y33X); and modifications to Trp64 (SEQ ID NO: 21-CCL2 W64X); wherein X is any amino acid excluding tyrosine, tryptophan, cysteine, and methionine.

SEQ ID NO: 36 represents a CCL expressed in humans. In some embodiments, SEQ ID NO: 36 comprises one or more mutations to Tyr36, Tyr51, and/or Trp82. In some embodiments, SEQ ID NO: 36 comprises any number of modifications, including but not limited to modifications at Tyr36 (CCL2 Y36X); modifications to Tyr51 (CCL2 Y5IX); and modifications to Trp82 (CCL2 W82X); wherein X is any amino acid excluding tyrosine, tryptophan, cysteine, and methionine.

This modification can further comprise any number of combinations of modifications, including but not limited to combined modifications to Tyr18 and Tyr33 (SEQ ID NO: 5 or SEQ ID NO: 22-Y18X and Y33X); combined modifications to Tyr18 and Trp64 (SEQ ID NO: 6 or SEQ ID NO: 23-Y18X and W64X); combined modifications to Tyr33 and Trp64 (SEQ ID NO: 7 or SEQ ID NO: 24-Y33X and W64X); and combined modifications to Tyr18, Tyr33, and Trp64 (SEQ ID NO: 8 or SEQ ID NO: 25-Y18X, Y33X, and W64X); wherein X is any amino acid excluding tyrosine, tryptophan, cysteine, and methionine. These sequences can also comprise other mutations, as described in detail below.

This modification can further comprise any number of combinations of modifications, including but not limited to combined modifications to Tyr36 and Tyr51; combined modifications to Tyr36 and Trp82; combined modifications to Tyr51 and Trp82; and combined modifications to Tyr36, Tyr51, and Trp82; wherein X is any amino acid excluding tyrosine, tryptophan, cysteine, and methionine.

In some embodiments, the Tyr is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Tyr is mutated into an alanine. In some embodiments, the Tyr is mutated into a glycine. In some embodiments, the Tyr is mutated into an isoleucine. In some embodiments, the Tyr is mutated into a leucine. In some embodiments, the Tyr is mutated into a proline. In some embodiments, the Tyr is mutated into a valine. In some embodiments, the Tyr is mutated into an aspartate or aspartic acid. In some embodiments, the Tyr is mutated into a glutamate or glutamic acid. In some embodiments, the Tyr is mutated into an arginine. In some embodiments, the Tyr is mutated into a histidine. In some embodiments, the Tyr is mutated into a lysine. In some embodiments, the Tyr is mutated into a serine. In some embodiments, the Tyr is mutated into a threonine. In some embodiments, the Tyr is mutated into an asparagine. In some embodiments, the Tyr is mutated into a phenylalanine. In some embodiments, the Tyr is mutated into a glutamine.

In another embodiment, the Trp is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Trp is mutated into an alanine. In some embodiments, the Trp is mutated into a glycine. In some embodiments, the Trp is mutated into an isoleucine. In some embodiments, the Trp is mutated into a leucine. In some embodiments, the Trp is mutated into a proline. In some embodiments, the Trp is mutated into a valine. In some embodiments, the Trp is mutated into an aspartate or aspartic acid. In some embodiments, the Trp is mutated into a glutamate or glutamic acid. In some embodiments, the Trp is mutated into an arginine. In some embodiments, the Trp is mutated into a histidine. In some embodiments, the Trp is mutated into a lysine. In some embodiments, the Trp is mutated into a serine. In some embodiments, the Trp is mutated into a threonine. In some embodiments, the Trp is mutated into an asparagine. In some embodiments, the Trp is mutated into a phenylalanine. In some embodiments, the Trp is mutated into a glutamine.

In another embodiment, the Met is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Met is mutated into an alanine. In some embodiments, the Met is mutated into a glycine. In some embodiments, the Met is mutated into an isoleucine. In some embodiments, the Met is mutated into a leucine. In some embodiments, the Met is mutated into a proline. In some embodiments, the Met is mutated into a valine. In some embodiments, the Met is mutated into an aspartate or aspartic acid. In some embodiments, the Met is mutated into a glutamate or glutamic acid. In some embodiments, the Met is mutated into an arginine. In some embodiments, the Met is mutated into a histidine. In some embodiments, the Met is mutated into a lysine. In some embodiments, the Met is mutated into a serine. In some embodiments, the Met is mutated into a threonine. In some embodiments, the Met is mutated into an asparagine. In some embodiments, the Met is mutated into a phenylalanine. In some embodiments, the Met is mutated into a glutamine.

In another embodiment, the Cys is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine. In some embodiments, the Cys is mutated into an alanine. In some embodiments, the Cys is mutated into a glycine. In some embodiments, the Cys is mutated into an isoleucine. In some embodiments, the Cys is mutated into a leucine. In some embodiments, the Cys is mutated into a proline. In some embodiments, the Cys is mutated into a valine. In some embodiments, the Cys is mutated into an aspartate or aspartic acid. In some embodiments, the Cys is mutated into a glutamate or glutamic acid. In some embodiments, the Cys is mutated into an arginine. In some embodiments, the Cys is mutated into a histidine. In some embodiments, the Cys is mutated into a lysine. In some embodiments, the Cys is mutated into a serine. In some embodiments, the Cys is mutated into a threonine. In some embodiments, the Cys is mutated into an asparagine. In some embodiments, the Cys is mutated into a phenylalanine. In some embodiments, the Cys is mutated into a glutamine.

In some embodiments, the nitration of the CCL2 is reduced or eliminated by at least 1% or more. In some embodiments, the nitration of the CCL2 is reduced or eliminated by 10% or more. In other embodiments, the nitration of the CCL2 is reduced or eliminated by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to a control.

In some embodiments, the nitration resistant CCL2 comprises an amino acid sequence comprising any one of SEQ ID NO: 2-8, or any variations thereof. In some embodiments, the nitration resistant CCL2 comprises SEQ ID NO: 2, or any variations thereof. In embodiments, the nitration resistant CCL2 comprises SEQ ID NO: 3, or any variations thereof. In some embodiments, the nitration resistant CCL2 comprises SEQ ID NO: 4, or any variations thereof. In some embodiments, the nitration resistant CCL2 comprises SEQ ID NO: 5, or any variations thereof. In some embodiments, the nitration resistant CCL2 comprises SEQ ID NO: 6, or any variations thereof. In some embodiments, the nitration resistant CCL2 comprises SEQ ID NO: 7, or any variation thereof. In some embodiments, the nitration resistant CCL2 comprises SEQ ID NO: 8, or any variation thereof.

In some embodiments, the nitration resistant CCL2 comprises an amino acid sequence comprising any one of SEQ ID NO: 19-25, or any variants thereof. In some embodiments, the nitration resistant CCL2 comprises SEQ ID NO: 19, or any variations thereof. In embodiments, the nitration resistant CCL2 comprises SEQ ID NO: 20, or any variations thereof. In some embodiments, the nitration resistant CCL2 comprises SEQ ID NO: 21, or any variations thereof. In some embodiments, the nitration resistant CCL2 comprises SEQ ID NO: 22, or any variations thereof. In some embodiments, the nitration resistant CCL2 comprises SEQ ID NO: 23, or any variations thereof. In some embodiments, the nitration resistant CCL2 comprises SEQ ID NO: 24, or any variation thereof. In some embodiments, the nitration resistant CCL2 comprises SEQ ID NO: 25, or any variation thereof.

### Expression Vectors

In one aspect, disclosed herein is an expression vector comprising a nucleic acid sequence encoding a nitration resistant C—C motif ligand 2 (CCL2) chemokine, wherein the nucleic acid sequence encodes one or more mutation of a tyrosine (Tyr), a tryptophan (Trp), a methionine (Met), or a cysteine (Cys) amino acid. In some embodiments, the vector encodes a recombinant CCL2 polypeptide, or a variant thereof.

In some embodiments, the nitration resistant CCL2 is encoded by a nucleic acid sequence comprising any one of 10-17, SEQ ID NO: 27-34, or any variations thereof. In some embodiments, the nitration resistant CCL2 is encoded by SEQ ID NO: 10, or any variations thereof. In some embodiments, the nitration resistant CCL2 is encoded by SEQ ID NO: 11, or any variations thereof. In some embodiments, the nitration resistant CCL2 is encoded by SEQ ID NO: 12, or any variations thereof. In some embodiments, the nitration resistant CCL2 is encoded by SEQ ID NO: 13, or any variations thereof. In some embodiments, the nitration resistant CCL2 is encoded by SEQ ID NO: 14, or any variations thereof. In some embodiments, the nitration resistant CCL2 is encoded by SEQ ID NO: 15, or any variations thereof. In some embodiments, the nitration resistant CCL2 is encoded by SEQ ID NO: 16, or any variations thereof. In some embodiments, the nitration resistant CCL2 is encoded by SEQ ID NO: 27, or any variations thereof. In some embodiments, the nitration resistant CCL2 is encoded by SEQ ID NO: 28, or any variations thereof. In some embodiments, the nitration resistant CCL2 is encoded by SEQ ID NO: 29, or any variations thereof. In some embodiments, the nitration resistant CCL2 is encoded by SEQ ID NO: 30, or any variations thereof. In some embodiments, the nitration resistant CCL2 is encoded by SEQ ID NO: 31, or any variations thereof. In some embodiments, the nitration resistant CCL2 is encoded by SEQ ID NO: 32, or any variations thereof. In some embodiments, the nitration resistant CCL2 is encoded by SEQ ID NO: 33, or any variations thereof. In some embodiments, the nitration resistant CCL2 is encoded by SEQ ID NO: 34, or any variations thereof.

In some embodiments, the nucleic acid sequence comprises at least 70% sequence identity to any one of SEQ ID NO: 10-17, SEQ ID NO: 27-34, or variants thereof. In some embodiments, the nucleic acid sequence comprises at least 80% sequence identity to any one of SEQ ID NO: 10-17, SEQ ID NO: 27-34, or variants thereof. In some embodiments, the nucleic acid sequence comprises at least 90% sequence identity to any one of SEQ ID NO: 10-17, SEQ ID NO: 27-34, or variants thereof. In some embodiments, the nucleic acid sequence comprises at least 95% sequence identity to any one of SEQ ID NO: 10-17, SEQ ID NO: 27-34, or variants thereof. In some embodiments, the nucleic acid sequence comprises at least 99% sequence identity to any one of SEQ ID NO: 10-17. SEQ ID NO: 27-34, or variants thereof. In some embodiments, the nucleic acid sequence comprises 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% sequence identity to any one of SEQ ID NO: 10-17, SEQ ID NO: 27-34, or variants thereof, or any percentage above, below, or in between the disclosed range.

In some embodiments, the nucleic acid sequence comprises any one of SEQ ID NO: 10-17, SEQ ID NO: 27-34, or variants thereof.

Also disclosed herein is SEQ ID NO: 10, which is the nucleic acid sequence representative of wild-type CCL2 in humans. Disclosed herein is SEQ ID NO: 27, which is the nucleic acid sequence representative of wild-type CCL2 in mice. Also disclosed are nucleic acid sequences which encode the above mutated amino acid sequences (SEQ ID NO: 2-8. SEQ ID NO: 19-25, or any variants thereof). The nucleic acid sequences comprising SEQ ID NO: 11-17, SEQ ID NO: 28-34, or any variants thereof, encode these mutated amino acid sequences.

In some embodiments, the recombinant CCL2 comprises SEQ ID NO: 9 (human), or any variants thereof, or SEQ ID NO: 26 (mouse), or any variants thereof.

As used herein, an “expression vector” or “vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In some embodiments, the recombinant CCL2 is expressed in an expression vector. In some embodiments, the recombinant CCL2 is expressed in a bacterial plasmid, or virus, a viral vector, or a nanoparticle. In some embodiments, the recombinant CCL2 is expressed in a pET29-NTEV plasmid.

### Methods of Treating Cancer

The present disclosure provides methods of treating, preventing, decreasing, reducing, and/or ameliorating cancer. The present disclosure also provides methods of reducing or eliminating nitration of a CCL2 chemokine using a nitration resistant CCL2 chemokine.

In one aspect, disclosed herein is a method of treating or preventing a cancer in a subject, the method comprising administering a pharmaceutically effective amount of a composition comprising a nitration resistant C—C motif ligand 2 (CCL2) chemokine and a pharmaceutically acceptable carrier comprising a salt, carrier, excipient, diluent, surfactant, buffer, or nanoparticle, wherein the nitration resistant CCL2 comprises one or more mutations of a tyrosine (Tyr), a tryptophan (Trp), a methionine (Met), or a cysteine (Cys) amino acid.

In one aspect, disclosed herein is a method of reducing or eliminating nitration of a CCL2 chemokine, the method comprising administering a pharmaceutically effective amount of a composition comprising a nitration resistant C—C motif ligand 2 (CCL2) chemokine and a pharmaceutically acceptable carrier comprising a salt, carrier, excipient, diluent, surfactant, buffer, or nanoparticle, wherein the nitration resistant CCL2 comprises one or more mutations of a tyrosine (Tyr), a tryptophan (Trp), a methionine (Met), or a cysteine (Cys) amino acid.

In some embodiments, the nitration resistant CCL2 outcompetes a native CCL2.

In some embodiments, the nitration resistant CCL2 recruits a CD8⁺ T cell or a monocytic myeloid cell. In some embodiments, the nitration resistant CCL2 recruits a CD4+ T cell. In some embodiments, the nitration resistant CCL2 recruits a B cell.

In some embodiments, the nitration resistant CCL2 is a recombinant CCL2. In some embodiments, the recombinant CCL2 comprises any of SEQ ID NO: 2-8 (in humans), SEQ ID NO: 18-25 (in mice), or any variants thereof. In some embodiments, the recombinant CCL2 is expressed in an expression vector of any preceding aspect. In some embodiments, the recombinant CCL2 is expression in a bacterial plasmid or a viral vector of any preceding aspect.

Non-limiting examples of cancer include acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma). Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLUSLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leionyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In some embodiments, the nitration resistant CCL2 is an anti-tumor chemokine. In some embodiments, the nitration resistant CCL2 cannot be nitrated at a tyrosine (Tyr), a tryptophan (Trp), a methionine (Met), or a cysteine (Cys) amino acid. In some embodiments, the nitration resistant CCL2 cannot be nitrated at a tyrosine. In some embodiments, the nitration resistant CCL2 cannot be nitrated at a tryptophan. In some embodiments, the nitration resistant CCL2 cannot be nitrated at a methionine. In some embodiments, the nitration resistant CCL2 cannot be nitrated at a cysteine. In some embodiments, the nitration resistant CCL2 cannot be nitrated at Tyr36, Tyr51, and/or Trp82. In some embodiments, the nitration resistant CCL2 cannot be nitrated at Tyr18, Tyr33, and/or Trp64.

In some embodiments, the nitration resistant CCL2 chemokine of any preceding aspect is further administered with an anti-cancer agent.

Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituxinab), BEXXAR (tositumomab)).

Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103. XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), Iloxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™ AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISELO), ENMD-2076, PCI-32765, AC220, dovitinib lactate (TK1258, CHIR-258), BIBW 2992 (TOVOK™). SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In some embodiments, the nitration resistant CCL2 chemokine of any preceding aspect is administered by an injection or by an instillation. In some embodiments, the injection is administered intravenously, intramuscularly, subcutaneously, or intraperitoneally. In some embodiments, the nitration resistant CCL2 chemokine is encapsulated or incorporated into a pharmaceutically acceptable carrier. “Pharmaceutically acceptable carrier” (sometimes referred to as a “carrier”) means a carrier or excipient that is useful in preparing a pharmaceutical composition that is generally safe and non-toxic, and includes a carrier that is acceptable for veterinary and/or human pharmaceutical or therapeutic use. The terms “carrier” or “pharmaceutically acceptable carrier” can include, but is not limited to, phosphate buffered saline solution, water, emulsions (such as an oil/water or water/oil emulsion) and/or various types of wetting agents. In some embodiments, the pharmaceutically acceptable carrier comprises a nanoparticle. In some embodiments, the pharmaceutically acceptable carrier comprises a lipid nanoparticle. In some embodiments, the nitration resistant CCL2 chemokine is prepared with a pharmaceutically acceptable salt, excipient, diluent, or surfactant. The preparation of pharmaceutically acceptable carriers and formulations containing these materials is described in, e.g., Remington's Pharmaceutical Sciences. 21st Edition, ed. University of the Sciences in Philadelphia, Lippincott. Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICT, Inc.; Bridgewater, New Jersey), polyethylene glycol (PEG), and PLURONICS™ (BASF; Florham Park, NJ).

The nitration resistant CCL2 chemokine of any preceding aspect may be administered in such amounts, time, and route deemed necessary in order to achieve the desired result. The exact amount of the nitration resistant CCL2 chemokine of any preceding aspect will vary from subject to subject, depending on the species, age, and general condition of the subject, the severity of the tumor(s), the particular composition, its mode of administration, its mode of activity, and the like. The nitration resistant CCL2 chemokine of any preceding aspect is preferably formulated in dosage unit form for ease of administration and uniformity of dosage. It will be understood, however, that the total daily usage of the nitration resistant CCL2 chemokine of any preceding aspect will be decided by the attending physician within the scope of sound medical judgment. The specific therapeutically effective dose level for any particular subject will depend upon a variety of factors including the cancer being treated and the severity of the cancer progression; the age, body weight, general health, sex and diet of the patient; the time of administration, route of administration, and rate of excretion of the nitration resistant CCL2 chemokine of any preceding aspect employed; the duration of the treatment; drugs used in combination or coincidental with the nitration resistant CCL2 chemokine of any preceding aspect employed; and like factors well known in the medical arts.

The nitration resistant CCL2 chemokine of any preceding aspect may be administered by any route. In some embodiments, the nitration resistant CCL2 chemokine is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the composition (e.g., its stability in the environment of the subject's body), the condition of the subject (e.g., whether the subject is able to tolerate administration), etc.

The exact amount of a nitration resistant CCL2 chemokine of any preceding aspect required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

In some embodiments, the subject is a mammal. In some embodiments, the subject is a mouse, a rabbit, a rat, a dog, a cat, a hamster, or a non-human primate.

In some embodiments, the subject is a human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

Herein, posttranslational nitration is shown to affect CCL2 function, but investigations on how this modification affects the activity of CCL2 in the tumor microenvironment—the effect of nitration on CCL2 mediated recruitment, activation, proliferation and cytotoxicity of immune cells and the direct effects on tumor cells—remains scarce. It has been shown that chemically nitrated recombinant (r) CCL2 fails to treat bladder cancer (BCa) and recruits significantly less CCR2+CD8+T cells but increased migration of monocytes to the bladder compared with unmodified CCL2 in a murine BCa model. Nitration of human rCCL2 was also shown to reduce CCL2's ability to induce T cell migration in transwell assays. Based on mass spectrometry data, peroxynitrate treated CCL2 was found to be nitrated at Tyrosine36 and 51 and Tryptophan 82 inhibiting recruitment of T cells in transwell assays. The efficacy of rCCL2 is improved when CCL2 is resistant to nitration (FIG. 1). Mutation of tyrosine and tryptophan amino acid residues of human rCCL2 increase rCCL2 mediated recruitment of T cells in BCa and improves treatment efficacy.

### Example 2

Herein, recruitment of immunosuppressive monocytic cells by CCL2 is shown to be promoted in breast, colon, glioblastoma, and prostate cancers. Thus, CCL2 blocking agents are under clinical investigation for treating solid tumors. However, CCL2 also recruits cytotoxic T cell, thereby contributing to antitumor immunity in some environments. The present disclosure demonstrates a protective role of CCL2 and CCR2 in the bladder. Recombinant CCL2 (rCCL2) is therapeutic, an effect which is lost when CCL2 is nitrated. Most chemokines, including CCL2, are thought to be constitutively active, and their activities not considered to be altered when post translationally modified. This is one of the critical reasons behind the failure of chemokines in clinical trials. Nitration of rCCL2 renders rCCL2 ineffective in bladder cancer and reduces the infiltration of T cells to the bladder. The nitration resistant CCL2 variant selectively recruits T cells to the bladder and emerge as treatment strategy in cancer.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
Quantification of Human CCL2 Clones

| | | Concentration of | | |
|---|---|---|---|---|
| | | 20 mL aliquots | | Number |
| Clone | CCL2 | (mg/mL), calculated | Concentration | of 20 mL |
| ID | Mutation | by BCA assay | in mM | aliquots |
| T31 | Wild-type | 4.42 | 486 | 14 |
| T32 | Y36F | 1.78 | 196 | 17 |
| T33 | Y36E | 4.46 | 493 | 36 |
| T34 | Y36I | 8.66 | 956 | 38 |
| T35 | Y36T | 4.08 | 452 | 38 |
| T36 | Y51F | 1.83 | 203 | 38 |
| T37 | Y51E | 1.67 | 184 | 18 |
| T38 | Y51I | 0.05 | 5.5 | 18 |
| | | (estimated by | | |
| | | SDS-PAGE) | | |
| T39 | Y51T | 0.71 | 79 | 18 |
| T40 | W82F | 3.19 | 353 | 19 |

TABLE 2
Quantification of Mouse CCL2 Clones

| | | Concentration of | | |
|---|---|---|---|---|
| | | 20 μl aliquots | | Number |
| Clone | CCL2 | (mg/ml), calculated | Concentration | of 20 mL |
| ID | Mutation | by BCA assay | in mM | aliquots |
| T58 | Wild-type | 1.38 | 155 | 24 |
| T59 | Y36F | 7.28 | 811 | 22 |
| T60 | Y36E | 10 | 1124 | 24 |
| T61 | Y36I | 12.4 | 1396 | 24 |
| T62 | Y36T | 5.4 | 609 | 33 |
| T63 | Y51F | 3.8 | 426 | 26 |
| T64 | Y51E | 6.65 | 747 | 24 |
| T65 | Y51I | 7.15 | 805 | 30 |
| T66 | Y51T | 1.1 | 124 | 21 |
| T67 | W82F | 4.66 | 524 | 21 |

SEQUENCES

```
  1. SEQ ID NO: 1 - Modified WT Human CCL2
GAMGSQPDAINAPVTCCYNFTNRKISVQRLASYRRITSSKCPKEAVIFKTIVAKEICADPKQ
KWVQDSMDHLDKQTQTPKT
  2. SEQ ID NO: 2 - Modified WT Human CCL2 Y18X
GAMGSQPDAINAPVTCCXNFTNRKISVQRLASYRRITSSKCPKEAVIFKTIVAKEICADPKQ
KWVQDSMDHLDKQTQTPKT
  3. SEQ ID NO: 3 - Modified WT Human CCL2 Y33X
GAMGSQPDAINAPVTCCYNFTNRKISVQRLASXRRITSSKCPKEAVIFKTIVAKEICADPKQ
KWVQDSMDHLDKQTQTPKT
  4. SEQ ID NO: 4 - Modified WT Human CCL2 W64X
GAMGSQPDAINAPVTCCYNFTNRKISVQRLASYRRITSSKCPKEAVIFKTIVAKEICADPKQ
KXVQDSMDHLDKQTQTPKT
  5. SEQ ID NO: 5 - Modified WT Human CCL2 Y18X and Y33X
GAMGSQPDAINAPVTCCXNFTNRKISVQRLASXRRITSSKCPKEAVIFKTIVAKEICADPKQ
KWVQDSMDHLDKQTQTPKT
  6. SEQ ID NO: 6 - Modified WT Human CCL2 Y18X and W64X
GAMGSQPDAINAPVTCCXNFTNRKISVQRLASYRRITSSKCPKEAVIFKTIVAKEICADPKQ
KXVQDSMDHLDKQTQTPKT
  7. SEQ ID NO: 7 - Modified WT Human CCL2 Y33X and W64X
GAMGSQPDAINAPVTCCYNFTNRKISVQRLASXRRITSSKCPKEAVIFKTIVAKEICADPKQ
KXVQDSMDHLDKQTQTPKT
  8. SEQ ID NO: 8 - Modified WT Human CCL2 Y18X, Y33X, and W64X
GAMGSQPDAINAPVTCCXNFTNRKISVQRLASXRRITSSKCPKEAVIFKTIVAKEICADPKQ
KXVQDSMDHLDKQTQTPKT
  9. SEQ ID NO: 9 - Full length Human WT CCL2 expressed in pET29-NTEV
MSYYHHHHHHDYDIPTTENLYFQGAMGSQPDAINAPVTCCYNFTNRKISVQRLASYRRITS
SKCPKEAVIFKTIVAKEICADPKQKWVQDSMDHLDKQTQTPKT
10. SEQ ID NO: 10 - WT Human CCL2 gene
GGCGCCATGGGCAGCCAGCCCGACGCCATCAACGCCCCCGTGACCTGCTGCTACAACT
TCACCAACAGGAAGATCAGCGTGCAGAGGCTGGCCAGCTACAGGAGGATCACCAGCA
GCAAGTGCCCCAAGGAGGCCGTGATCTTCAAGACCATCGTGGCCAAGGAGATCTGCGC
CGACCCCAAGCAGAAGTGGGTGCAGGACAGCATGGACCACCTGGACAAGCAGACCCA
GACCCCCAAGACC
11. SEQ ID NO: 11 - WT Human CCL2 Y18X gene
GGCGCCATGGGCAGCCAGCCCGACGCCATCAACGCCCCCGTGACCTGCTGCNNNAACT
TCACCAACAGGAAGATCAGCGTGCAGAGGCTGGCCAGCTACAGGAGGATCACCAGCA
GCAAGTGCCCCAAGGAGGCCGTGATCTTCAAGACCATCGTGGCCAAGGAGATCTGCGC
CGACCCCAAGCAGAAGTGGGTGCAGGACAGCATGGACCACCTGGACAAGCAGACCCA
GACCCCCAAGACC
12. SEQ ID NO: 12 - WT Human CCL2 Y33X gene
GGCGCCATGGGCAGCCAGCCCGACGCCATCAACGCCCCCGTGACCTGCTGCTACAACT
TCACCAACAGGAAGATCAGCGTGCAGAGGCTGGCCAGCNNNAGGAGGATCACCAGCA
GCAAGTGCCCCAAGGAGGCCGTGATCTTCAAGACCATCGTGGCCAAGGAGATCTGCGC
CGACCCCAAGCAGAAGTGGGTGCAGGACAGCATGGACCACCTGGACAAGCAGACCCA
GACCCCCAAGACC
13. SEQ ID NO: 13 - WT Human CCL2 W64X gene
GGCGCCATGGGCAGCCAGCCCGACGCCATCAACGCCCCCGTGACCTGCTGCTACAACT
TCACCAACAGGAAGATCAGCGTGCAGAGGCTGGCCAGCTACAGGAGGATCACCAGCA
GCAAGTGCCCCAAGGAGGCCGTGATCTTCAAGACCATCGTGGCCAAGGAGATCTGCGC
CGACCCCAAGCAGAAGNNNGTGCAGGACAGCATGGACCACCTGGACAAGCAGACCCA
GACCCCCAAGACC
14. SEQ ID NO: 14 - WT Human CCL2 Y18X and Y33X gene
GGCGCCATGGGCAGCCAGCCCGACGCCATCAACGCCCCCGTGACCTGCTGCNNNAACT
TCACCAACAGGAAGATCAGCGTGCAGAGGCTGGCCAGCNNNAGGAGGATCACCAGCA
GCAAGTGCCCCAAGGAGGCCGTGATCTTCAAGACCATCGTGGCCAAGGAGATCTGCGC
CGACCCCAAGCAGAAGTGGGTGCAGGACAGCATGGACCACCTGGACAAGCAGACCCA
GACCCCCAAGACC
15. SEQ ID NO: 15 - WT Human CCL2 Y18X and W64X gene
GGCGCCATGGGCAGCCAGCCCGACGCCATCAACGCCCCCGTGACCTGCTGCNNNAACT
TCACCAACAGGAAGATCAGCGTGCAGAGGCTGGCCAGCTACAGGAGGATCACCAGCA
GCAAGTGCCCCAAGGAGGCCGTGATCTTCAAGACCATCGTGGCCAAGGAGATCTGCGC
CGACCCCAAGCAGAAGNNNGTGCAGGACAGCATGGACCACCTGGACAAGCAGACCCA
GACCCCCAAGACC
16. SEQ ID NO: 16 - WT Human CCL2 Y33X and W64X gene
GGCGCCATGGGCAGCCAGCCCGACGCCATCAACGCCCCCGTGACCTGCTGCTACAACT
TCACCAACAGGAAGATCAGCGTGCAGAGGCTGGCCAGCNNNAGGAGGATCACCAGCA
GCAAGTGCCCCAAGGAGGCCGTGATCTTCAAGACCATCGTGGCCAAGGAGATCTGCGC
CGACCCCAAGCAGAAGNNNGTGCAGGACAGCATGGACCACCTGGACAAGCAGACCCA
GACCCCCAAGACC
17. SEQ ID NO: 17 - WT Human CCL2 Y18X, Y33X, and W64X gene
GGCGCCATGGGCAGCCAGCCCGACGCCATCAACGCCCCCGTGACCTGCTGCNNNAACT
TCACCAACAGGAAGATCAGCGTGCAGAGGCTGGCCAGCNNNAGGAGGATCACCAGCA
GCAAGTGCCCCAAGGAGGCCGTGATCTTCAAGACCATCGTGGCCAAGGAGATCTGCGC
CGACCCCAAGCAGAAGNNNGTGCAGGACAGCATGGACCACCTGGACAAGCAGACCCA
GACCCCCAAGACC
18. SEQ ID NO: 18 - Modified Wild-type Mouse CCL2
GAMGSQPDAVNAPLTCCYSFTSKMIPMSRLESYKRITSSRCPKEAVVFVTKLKREVCADPK
KEWVQTYIKNLDRNQMR
19. SEQ ID NO: 19 - Modified Mouse CCL2 Y18X
GAMGSQPDAVNAPLTCCXSFTSKMIPMSRLESYKRITSSRCPKEAVVFVTKLKREVCADPK
KEWVQTYIKNLDRNQMR
20. SEQ ID NO: 20 - Modified Mouse CCL2 Y33X
GAMGSQPDAVNAPLTCCYSFTSKMIPMSRLESXKRITSSRCPKEAVVFVTKLKREVCADPK
KEWVQTYIKNLDRNQMR
21. SEQ ID NO: 21 - Modified Mouse CCL2 W64X
GAMGSQPDAVNAPLTCCYSFTSKMIPMSRLESYKRITSSRCPKEAVVFVTKLKREVCADPK
KEXVQTYIKNLDRNQMR
22. SEQ ID NO: 22 - Modified Mouse CCL2 Y18X and Y33X
GAMGSQPDAVNAPLTCCXSFTSKMIPMSRLESXKRITSSRCPKEAVVFVTKLKREVCADPK
KEWVQTYIKNLDRNQMR
23. SEQ ID NO: 23 - Modified Mouse CCL2 Y36X and W82X
GAMGSQPDAVNAPLTCCXSFTSKMIPMSRLESYKRITSSRCPKEAVVFVTKLKREVCADPK
KEXVQTYIKNLDRNQMR
24. SEQ ID NO: 24 - Modified Mouse CCL2 Y51X and W82X
GAMGSQPDAVNAPLTCCYSFTSKMIPMSRLESXKRITSSRCPKEAVVFVTKLKREVCADPK
KEXVQTYIKNLDRNQMR
25. SEQ ID NO: 25 - Modified Mouse CCL2 Y36X, Y51X, and W82X
GAMGSQPDAVNAPLTCCXSFTSKMIPMSRLESXKRITSSRCPKEAVVFVTKLKREVCADPK
KEXVQTYIKNLDRNQMR
26. SEQ ID NO: 26 - Full length Mouse Wildtype CCL2 expressed in pET29-NTEV
MSYYHHHHHHDYDIPTTENLYFQGAMGSQPDAVNAPLTCCYSFTSKMIPMSRLESYKRITS
SRCPKEAVVFVTKLKREVCADPKKEWVQTYIKNLDRNQMR
27. SEQ ID NO: 27 - Mouse Wildtype CCL2
GGCGCCATGGGCAGCCAGCCCGACGCCGTGAACGCCCCCCTGACCTGCTGCTACAGCT
TCACCAGCAAGATGATCCCCATGAGCAGGCTGGAGAGCTACAAGAGGATCACCAGCA
GCAGGTGCCCCAAGGAGGCCGTGGTGTTCGTGACCAAGCTGAAGAGGGAGGTGTGCGC
CGACCCCAAGAAGGAGTGGGTGCAGACCTACATCAAGAACCTGGACAGGAACCAGAT
GAGG
28. SEQ ID NO: 28 - Mouse CCL2 Y18X
GGCGCCATGGGCAGCCAGCCCGACGCCGTGAACGCCCCCCTGACCTGCTGCNNNAGCT
TCACCAGCAAGATGATCCCCATGAGCAGGCTGGAGAGCTACAAGAGGATCACCAGCA
GCAGGTGCCCCAAGGAGGCCGTGGTGTTCGTGACCAAGCTGAAGAGGGAGGTGTGCGC
CGACCCCAAGAAGGAGTGGGTGCAGACCTACATCAAGAACCTGGACAGGAACCAGAT
GAGG
29. SEQ ID NO: 29 - Mouse CCL2 Y33X
GGCGCCATGGGCAGCCAGCCCGACGCCGTGAACGCCCCCCTGACCTGCTGCTACAGCT
TCACCAGCAAGATGATCCCCATGAGCAGGCTGGAGAGCNNNAAGAGGATCACCAGCA
GCAGGTGCCCCAAGGAGGCCGTGGTGTTCGTGACCAAGCTGAAGAGGGAGGTGTGCGC
CGACCCCAAGAAGGAGTGGGTGCAGACCTACATCAAGAACCTGGACAGGAACCAGAT
GAGG
30. SEQ ID NO: 30 - Mouse CCL2 W64X
GGCGCCATGGGCAGCCAGCCCGACGCCGTGAACGCCCCCCTGACCTGCTGCTACAGCT
TCACCAGCAAGATGATCCCCATGAGCAGGCTGGAGAGCTACAAGAGGATCACCAGCA
GCAGGTGCCCCAAGGAGGCCGTGGTGTTCGTGACCAAGCTGAAGAGGGAGGTGTGCGC
CGACCCCAAGAAGGAGNNNGTGCAGACCTACATCAAGAACCTGGACAGGAACCAGAT
GAGG
31. SEQ ID NO: 31 - Mouse CCL2 Y18X and Y33X
GGCGCCATGGGCAGCCAGCCCGACGCCGTGAACGCCCCCCTGACCTGCTGCNNNAGCT
TCACCAGCAAGATGATCCCCATGAGCAGGCTGGAGAGCNNNAAGAGGATCACCAGCA
GCAGGTGCCCCAAGGAGGCCGTGGTGTTCGTGACCAAGCTGAAGAGGGAGGTGTGCGC
CGACCCCAAGAAGGAGTGGGTGCAGACCTACATCAAGAACCTGGACAGGAACCAGAT
GAGG
32. SEQ ID NO: 32 - Mouse CCL2 Y18X and W64X
GGCGCCATGGGCAGCCAGCCCGACGCCGTGAACGCCCCCCTGACCTGCTGCNNNAGCT
TCACCAGCAAGATGATCCCCATGAGCAGGCTGGAGAGCTACAAGAGGATCACCAGCA
GCAGGTGCCCCAAGGAGGCCGTGGTGTTCGTGACCAAGCTGAAGAGGGAGGTGTGCGC
CGACCCCAAGAAGGAGNNNGTGCAGACCTACATCAAGAACCTGGACAGGAACCAGAT
GAGG
33. SEQ ID NO: 33 - Mouse CCL2 Y33X and W64X
GGCGCCATGGGCAGCCAGCCCGACGCCGTGAACGCCCCCCTGACCTGCTGCTACAGCT
TCACCAGCAAGATGATCCCCATGAGCAGGCTGGAGAGCNNNAAGAGGATCACCAGCA
GCAGGTGCCCCAAGGAGGCCGTGGTGTTCGTGACCAAGCTGAAGAGGGAGGTGTGCGC
CGACCCCAAGAAGGAGNNNGTGCAGACCTACATCAAGAACCTGGACAGGAACCAGAT
GAGG
34. SEQ ID NO: 34 - Mouse CCL2 Y18X, Y33X, and W64X
GGCGCCATGGGCAGCCAGCCCGACGCCGTGAACGCCCCCCTGACCTGCTGCNNNAGCT
TCACCAGCAAGATGATCCCCATGAGCAGGCTGGAGAGCNNNAAGAGGATCACCAGCA
GCAGGTGCCCCAAGGAGGCCGTGGTGTTCGTGACCAAGCTGAAGAGGGAGGTGTGCGC
CGACCCCAAGAAGGAGNNNGTGCAGACCTACATCAAGAACCTGGACAGGAACCAGAT
GAGG
35. SEQ ID NO: 35 - Full length Human WT CCL2
MKVSAALLCLLLIAATFIPQGLAQPDAINAPVTCCYNFTNRKISVQRLASYRRITSSKCPKEA
VIFKTIVAKEICADPKQKWVQDSMDHLDKQTQTPKT
36. SEQ ID NO: 36 - Full length Mouse WT CCL2
MQVPVMLLGLLFTVAGWSIHVLAQPDAVNAPLTCCYSFTSKMIPMSRLESYKRITSSRCPK
EAVVFVTKLKREVCADPKKEWVQTYIKNLDRNQMRSEPTTLFKTASALRSSAPLNVKLTR
KSEANASTTESTTTSSTSVGVTSVTVN
wherein X = any amino acid excluding tyrosine, tryptophan, cysteine, and methionine; and N = any nucleotide.
```

1. A composition comprising a nitration resistant C—C motif ligand 2 (CCL2) chemokine and a pharmaceutically acceptable salt, carrier, excipient, diluent, or surfactant, wherein the nitration resistant CCL2 comprises one or more mutations of a tyrosine (Tyr), a tryptophan (Trp), a methionine (Met), or a cysteine (Cys) amino acid.

2. The composition of claim 1, wherein the nitration resistant CCL2 chemokine comprises the one or more mutation at Tyr18, Tyr33, or Trp64 of SEQ ID NO: 1 or 18.

3. The composition of claim 1, wherein the nitration resistant CCL2 chemokine comprises the one or more mutation at Tyr36, Tyr51, or Trp82 of SEQ ID NO: 9 or 26.

4. The composition of claim 1, wherein the nitration resistant CCL2 chemokine comprises at least 70% sequence identity to any one of SEQ ID NO: 2-8, SEQ ID NO: 19-25, or variants thereof.

5. The composition of claim 1, wherein the nitration resistant CCL2 chemokine comprises at least 80% sequence identity to any one of SEQ ID NO: 2-8, SEQ ID NO: 19-25, or variants thereof.

6. The composition of claim 1, wherein the nitration resistant CCL2 chemokine comprises at least 90% sequence identity to any one of SEQ ID NO: 2-8, SEQ ID NO: 19-25, or variants thereof.

7. The composition of claim 1, wherein the nitration resistant CCL2 chemokine comprises any one of SEQ ID NO: 2-8, SEQ ID NO: 19-25, or variants thereof.

8. The composition of claim 1, wherein the Tyr is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine.

9. The composition of claim 1, wherein the Trp is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine.

10. The composition of claim 1, wherein the Met is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine.

11. The composition of claim 1, wherein the Cys is mutated into any one amino acid selected from the group consisting of alanine, glycine, isoleucine, leucine, proline, valine, aspartate, glutamate, arginine, histidine, lysine, serine, threonine, asparagine, phenylalanine, and glutamine.

12. An expression vector comprising a nucleic acid sequence encoding a nitration resistant C—C motif ligand 2 (CCL2) chemokine, wherein the nucleic acid sequence encodes one or more mutation of a tyrosine (Tyr), a tryptophan (Trp), a methionine (Met), or a cysteine (Cys) amino acid.

13. The expression vector of claim 12, wherein the nucleic acid sequence comprises at least 70% sequence identity to any one of SEQ ID NO: 11-17, SEQ ID NO: 28-34, or variants thereof.

14. The expression vector of claim 12, wherein the nucleic acid sequence comprises at least 80% sequence identity to any one of SEQ ID NO: 11-17, SEQ ID NO: 28-34, or variants thereof.

15. The expression vector of claim 12, wherein the nucleic acid sequence comprises at least 90% sequence identity to any one of SEQ ID NO: 11-17, SEQ ID NO: 28-34, or variants thereof.

16. The expression vector of claim 12, wherein the nucleic acid sequence comprises any one of SEQ ID NO: 11-17, SEQ ID NO: 28-34, or variants thereof.

17. A method of treating or preventing a cancer in a subject, the method comprising administering a pharmaceutically effective amount of a composition comprising a nitration resistant C—C motif ligand 2 (CCL2) chemokine and a pharmaceutically acceptable carrier comprising a salt, carrier, excipient, diluent, surfactant, buffer, or nanoparticle, wherein the nitration resistant CCL2 comprises one or more mutations of a tyrosine (Tyr), a tryptophan (Trp), a methionine (Met), or a cysteine (Cys) amino acid.

18-32. (canceled)


SEQUENCE LISTING (36 sequences)

SEQ ID NO: 1 - protein, 81 aa, Homo sapiens
  REGION 1..81: note = Description of sequence: Modified WT Human CCL2
  DOMAIN 1..5: note = protein tag amino acids
GAMGSQPDAINAPVTCCYNFTNRKISVQRLASYRRITSSKCPKEAVIFKTIVAKEICADPKQKWVQDSMDHLDKQTQTPKT

SEQ ID NO: 2 - protein, 81 aa, Homo sapiens
  REGION 1..81: note = Description of sequence: Modified WT Human CCL2
  DOMAIN 1..5: note = protein tag amino acids
  VARIANT 18: note = X represents any amino acid excluding tyrosine tryptophan cysteine and methionine
GAMGSQPDAINAPVTCCXNFTNRKISVQRLASYRRITSSKCPKEAVIFKTIVAKEICADPKQKWVQDSMDHLDKQTQTPKT

SEQ ID NO: 3 - protein, 81 aa, Homo sapiens
  REGION 1..81: note = Description of sequence: Modified WT Human CCL2
  DOMAIN 1..5: note = protein tag amino acids
  VARIANT 33: note = X represents any amino acid excluding tyrosine tryptophan cysteine and methionine
GAMGSQPDAINAPVTCCYNFTNRKISVQRLASXRRITSSKCPKEAVIFKTIVAKEICADPKQKWVQDSMDHLDKQTQTPKT

SEQ ID NO: 4 - protein, 81 aa, Homo sapiens
  REGION 1..81: note = Description of sequence: Modified WT Human CCL2
  DOMAIN 1..5: note = protein tag amino acids
  VARIANT 64: note = X represents any amino acid excluding tyrosine tryptophan cysteine and methionine
GAMGSQPDAINAPVTCCYNFTNRKISVQRLASYRRITSSKCPKEAVIFKTIVAKEICADPKQKXVQDSMDHLDKQTQTPKT

SEQ ID NO: 5 - protein, 81 aa, Homo sapiens
  REGION 1..81: note = Description of sequence: Modified WT Human CCL2
  DOMAIN 1..5: note = protein tag amino acids
  VARIANT 18: note = X represents any amino acid excluding tyrosine tryptophan cysteine and methionine
  VARIANT 33: note = X represents any amino acid excluding tyrosine tryptophan cysteine and methionine
GAMGSQPDAINAPVTCCXNFTNRKISVQRLASXRRITSSKCPKEAVIFKTIVAKEICADPKQKWVQDSMDHLDKQTQTPKT

SEQ ID NO: 6 - protein, 81 aa, Homo sapiens
  REGION 1..81: note = Description of sequence: Modified WT Human CCL2
  DOMAIN 1..5: note = protein tag amino acids
  VARIANT 18: note = X represents any amino acid excluding tyrosine tryptophan cysteine and methionine
  VARIANT 64: note = X represents any amino acid excluding tyrosine tryptophan cysteine and methionine
GAMGSQPDAINAPVTCCXNFTNRKISVQRLASYRRITSSKCPKEAVIFKTIVAKEICADPKQKXVQDSMDHLDKQTQTPKT

SEQ ID NO: 7 - protein, 81 aa, Homo sapiens
  REGION 1..81: note = Description of sequence: Modified WT Human CCL2
  DOMAIN 1..5: note = protein tag amino acids
  VARIANT 33: note = X represents any amino acid excluding tyrosine tryptophan cysteine and methionine
  VARIANT 64: note = X represents any amino acid excluding tyrosine tryptophan cysteine and methionine
GAMGSQPDAINAPVTCCYNFTNRKISVQRLASXRRITSSKCPKEAVIFKTIVAKEICADPKQKXVQDSMDHLDKQTQTPKT

SEQ ID NO: 8 - protein, 81 aa, Homo sapiens
  REGION 1..81: note = Description of sequence: Modified WT Human CCL2
  DOMAIN 1..5: note = protein tag amino acids
  VARIANT 18: note = X represents any amino acid excluding tyrosine tryptophan cysteine and methionine
  VARIANT 33: note = X represents any amino acid excluding tyrosine tryptophan cysteine and methionine
  VARIANT 64: note = X represents any amino acid excluding tyrosine tryptophan cysteine and methionine
GAMGSQPDAINAPVTCCXNFTNRKISVQRLASXRRITSSKCPKEAVIFKTIVAKEICADPKQKXVQDSMDHLDKQTQTPKT

SEQ ID NO: 9 - protein, 104 aa, Homo sapiens
  REGION 1..104: note = Description of sequence: Full length Human WT CCL2 expressed in pET29-NTEV
MSYYHHHHHHDYDIPTTENLYFQGAMGSQPDAINAPVTCCYNFTNRKISVQRLASYRRITSSKCPKEAVIFKTIVAKEICADPKQKWVQDSMDHLDKQTQTPKT

SEQ ID NO: 10 - DNA, 243 bp, Homo sapiens
  misc_feature 1..243: note = Description of sequence: WT Human CCL2 gene
  source 1..243: mol_type = genomic DNA
ggcgccatgggcagccagcccgacgccatcaacgcccccgtgacctgctgctacaacttcaccaacaggaagatcagcgtgcagaggctggccagctacaggaggatcaccagcagcaagtgccccaaggaggccgtgatcttcaagaccatcgtggccaaggagatctgcgccgaccccaagcagaagtgggtgcaggacagcatggaccacctggacaagcagacccagacccccaagacc

SEQ ID NO: 11 - DNA, 243 bp, Homo sapiens
  misc_feature 1..243: note = Description of sequence: WT Human CCL2 Y18X gene
  source 1..243: mol_type = genomic DNA
  variation 52..54: note = N represents any nucleotide
ggcgccatgggcagccagcccgacgccatcaacgcccccgtgacctgctgcnnnaacttcaccaacaggaagatcagcgtgcagaggctggccagctacaggaggatcaccagcagcaagtgccccaaggaggccgtgatcttcaagaccatcgtggccaaggagatctgcgccgaccccaagcagaagtgggtgcaggacagcatggaccacctggacaagcagacccagacccccaagacc

SEQ ID NO: 12 - DNA, 243 bp, Homo sapiens
  misc_feature 1..243: note = Description of sequence: WT Human CCL2 Y33X gene
  source 1..243: mol_type = genomic DNA
  variation 97..99: note = N represents any nucleotide
ggcgccatgggcagccagcccgacgccatcaacgcccccgtgacctgctgctacaacttcaccaacaggaagatcagcgtgcagaggctggccagcnnnaggaggatcaccagcagcaagtgccccaaggaggccgtgatcttcaagaccatcgtggccaaggagatctgcgccgaccccaagcagaagtgggtgcaggacagcatggaccacctggacaagcagacccagacccccaagacc

SEQ ID NO: 13 - DNA, 243 bp, Homo sapiens
  misc_feature 1..243: note = Description of sequence: WT Human CCL2 W64X gene
  source 1..243: mol_type = genomic DNA
  variation 190..192: note = N represents any nucleotide
ggcgccatgggcagccagcccgacgccatcaacgcccccgtgacctgctgctacaacttcaccaacaggaagatcagcgtgcagaggctggccagctacaggaggatcaccagcagcaagtgccccaaggaggccgtgatcttcaagaccatcgtggccaaggagatctgcgccgaccccaagcagaagnnngtgcaggacagcatggaccacctggacaagcagacccagacccccaagacc

SEQ ID NO: 14 - DNA, 243 bp, Homo sapiens
  misc_feature 1..243: note = Description of sequence: WT Human CCL2 Y18X and Y33X gene
  source 1..243: mol_type = genomic DNA
  variation 52..54: note = N represents any nucleotide
  variation 97..99: note = N represents any nucleotide
ggcgccatgggcagccagcccgacgccatcaacgcccccgtgacctgctgcnnnaacttcaccaacaggaagatcagcgtgcagaggctggccagcnnnaggaggatcaccagcagcaagtgccccaaggaggccgtgatcttcaagaccatcgtggccaaggagatctgcgccgaccccaagcagaagtgggtgcaggacagcatggaccacctggacaagcagacccagacccccaagacc

SEQ ID NO: 15 - DNA, 243 bp, Homo sapiens
  misc_feature 1..243: note = Description of sequence: WT Human CCL2 Y18X and W64X gene
  source 1..243: mol_type = genomic DNA
  variation 52..54: note = N represents any nucleotide
  variation 190..192: note = N represents any nucleotide
ggcgccatgggcagccagcccgacgccatcaacgcccccgtgacctgctgcnnnaacttcaccaacaggaagatcagcgtgcagaggctggccagctacaggaggatcaccagcagcaagtgccccaaggaggccgtgatcttcaagaccatcgtggccaaggagatctgcgccgaccccaagcagaagnnngtgcaggacagcatggaccacctggacaagcagacccagacccccaagacc

SEQ ID NO: 16 - DNA, 243 bp, Homo sapiens
  misc_feature 1..243: note = Description of sequence: WT Human CCL2 Y33X and W64X gene
  source 1..243: mol_type = genomic DNA
  variation 97..99: note = N represents any nucleotide
  variation 190..192: note = N represents any nucleotide
ggcgccatgggcagccagcccgacgccatcaacgcccccgtgacctgctgctacaacttcaccaacaggaagatcagcgtgcagaggctggccagcnnnaggaggatcaccagcagcaagtgccccaaggaggccgtgatcttcaagaccatcgtggccaaggagatctgcgccgaccccaagcagaagnnngtgcaggacagcatggaccacctggacaagcagacccagacccccaagacc

SEQ ID NO: 17 - DNA, 243 bp, Homo sapiens
  misc_feature 1..243: note = Description of sequence: WT Human CCL2 Y18X Y33X and W64X gene
  source 1..243: mol_type = genomic DNA
  variation 52..54: note = N represents any nucleotide
  variation 97..99: note = N represents any nucleotide
  variation 190..192: note = N represents any nucleotide
ggcgccatgggcagccagcccgacgccatcaacgcccccgtgacctgctgcnnnaacttcaccaacaggaagatcagcgtgcagaggctggccagcnnnaggaggatcaccagcagcaagtgccccaaggaggccgtgatcttcaagaccatcgtggccaaggagatctgcgccgaccccaagcagaagnnngtgcaggacagcatggaccacctggacaagcagacccagacccccaagacc

SEQ ID NO: 18 - protein, 78 aa, Mus musculus
  REGION 1..78: note = Description of sequence: Modified Wild-type Mouse CCL2
  DOMAIN 1..5: note = protein tag amino acids
GAMGSQPDAVNAPLTCCYSFTSKMIPMSRLESYKRITSSRCPKEAVVFVTKLKREVCADPKKEWVQTYIKNLDRNQMR

SEQ ID NO: 19 - protein, 78 aa, Mus musculus
  REGION 1..78: note = Description of sequence: Modified Mouse CCL2 Y18X
  DOMAIN 1..5: note = protein tag amino acids
  VARIANT 18: note = X represents any amino acid excluding tyrosine tryptophan cysteine and methionine
GAMGSQPDAVNAPLTCCXSFTSKMIPMSRLESYKRITSSRCPKEAVVFVTKLKREVCADPKKEWVQTYIKNLDRNQMR

SEQ ID NO: 20 - protein, 78 aa, Mus musculus
  REGION 1..78: note = Description of sequence: Modified Mouse CCL2 Y33X
  DOMAIN 1..5: note = protein tag amino acids
  VARIANT 33: note = X represents any amino acid excluding tyrosine tryptophan cysteine and methionine
GAMGSQPDAVNAPLTCCYSFTSKMIPMSRLESXKRITSSRCPKEAVVFVTKLKREVCADPKKEWVQTYIKNLDRNQMR

SEQ ID NO: 21 - protein, 78 aa, Mus musculus
  REGION 1..78: note = Description of sequence: Modified Mouse CCL2 W64X
  DOMAIN 1..5: note = protein tag amino acids
  VARIANT 64: note = X represents any amino acid excluding tyrosine tryptophan cysteine and methionine
GAMGSQPDAVNAPLTCCYSFTSKMIPMSRLESYKRITSSRCPKEAVVFVTKLKREVCADPKKEXVQTYIKNLDRNQMR

SEQ ID NO: 22 - protein, 78 aa, Mus musculus
  REGION 1..78: note = Description of sequence: Modified Mouse CCL2 Y18X and Y33X
  DOMAIN 1..5: note = protein tag amino acids
  VARIANT 18: note = X represents any amino acid excluding tyrosine tryptophan cysteine and methionine
  VARIANT 33: note = X represents any amino acid excluding tyrosine tryptophan cysteine and methionine
GAMGSQPDAVNAPLTCCXSFTSKMIPMSRLESXKRITSSRCPKEAVVFVTKLKREVCADPKKEWVQTYIKNLDRNQMR

SEQ ID NO: 23 - protein, 78 aa, Mus musculus
  REGION 1..78: note = Description of sequence: Modified Mouse CCL2 Y36X and W82X
  DOMAIN 1..5: note = protein tag amino acids
  VARIANT 18: note = X represents any amino acid excluding tyrosine tryptophan cysteine and methionine
  VARIANT 64: note = X represents any amino acid excluding tyrosine tryptophan cysteine and methionine
GAMGSQPDAVNAPLTCCXSFTSKMIPMSRLESYKRITSSRCPKEAVVFVTKLKREVCADPKKEXVQTYIKNLDRNQMR

SEQ ID NO: 24 - protein, 78 aa, Mus musculus
  REGION 1..78: note = Description of sequence: Modified Mouse CCL2 Y51X and W82X
  DOMAIN 1..5: note = protein tag amino acids
  VARIANT 33: note = X represents any amino acid excluding tyrosine tryptophan cysteine and methionine
  VARIANT 64: note = X represents any amino acid excluding tyrosine tryptophan cysteine and methionine
GAMGSQPDAVNAPLTCCYSFTSKMIPMSRLESXKRITSSRCPKEAVVFVTKLKREVCADPKKEXVQTYIKNLDRNQMR

SEQ ID NO: 25 - protein, 78 aa, Mus musculus
  REGION 1..78: note = Description of sequence: Modified Mouse CCL2 Y36X Y51X and W82X
  DOMAIN 1..5: note = protein tag amino acids
  VARIANT 18: note = X represents any amino acid excluding tyrosine tryptophan cysteine and methionine
  VARIANT 33: note = X represents any amino acid excluding tyrosine tryptophan cysteine and methionine
  VARIANT 64: note = X represents any amino acid excluding tyrosine tryptophan cysteine and methionine
GAMGSQPDAVNAPLTCCXSFTSKMIPMSRLESXKRITSSRCPKEAVVFVTKLKREVCADPKKEXVQTYIKNLDRNQMR

SEQ ID NO: 26 - protein, 101 aa, Mus musculus
  REGION 1..101: note = Description of sequence: Full length Mouse Wildtype CCL2 expressed in pET29-NTEV
MSYYHHHHHHDYDIPTTENLYFQGAMGSQPDAVNAPLTCCYSFTSKMIPMSRLESYKRITSSRCPKEAVVFVTKLKREVCADPKKEWVQTYIKNLDRNQMR

SEQ ID NO: 27 - DNA, 234 bp, Mus musculus
  misc_feature 1..234: note = Description of sequence: Mouse Wildtype CCL2
  source 1..234: mol_type = genomic DNA
ggcgccatgggcagccagcccgacgccgtgaacgcccccctgacctgctgctacagcttcaccagcaagatgatccccatgagcaggctggagagctacaagaggatcaccagcagcaggtgccccaaggaggccgtggtgttcgtgaccaagctgaagagggaggtgtgcgccgaccccaagaaggagtgggtgcagacctacatcaagaacctggacaggaaccagatgagg

SEQ ID NO: 28 - DNA, 234 bp, Mus musculus
  misc_feature 1..234: note = Description of sequence: Mouse CCL2 Y18X
  source 1..234: mol_type = genomic DNA
  variation 52..54: note = N represents any nucleotide
ggcgccatgggcagccagcccgacgccgtgaacgcccccctgacctgctgcnnnagcttcaccagcaagatgatccccatgagcaggctggagagctacaagaggatcaccagcagcaggtgccccaaggaggccgtggtgttcgtgaccaagctgaagagggaggtgtgcgccgaccccaagaaggagtgggtgcagacctacatcaagaacctggacaggaaccagatgagg

SEQ ID NO: 29 - DNA, 234 bp, Mus musculus
  misc_feature 1..234: note = Description of sequence: Mouse CCL2 Y33X
  source 1..234: mol_type = genomic DNA
  variation 97..99: note = N represents any nucleotide
ggcgccatgggcagccagcccgacgccgtgaacgcccccctgacctgctgctacagcttcaccagcaagatgatccccatgagcaggctggagagcnnnaagaggatcaccagcagcaggtgccccaaggaggccgtggtgttcgtgaccaagctgaagagggaggtgtgcgccgaccccaagaaggagtgggtgcagacctacatcaagaacctggacaggaaccagatgagg

SEQ ID NO: 30 - DNA, 234 bp, Mus musculus
  misc_feature 1..234: note = Description of sequence: Mouse CCL2 W64X
  source 1..234: mol_type = genomic DNA
  variation 190..192: note = N represents any nucleotide
ggcgccatgggcagccagcccgacgccgtgaacgcccccctgacctgctgctacagcttcaccagcaagatgatccccatgagcaggctggagagctacaagaggatcaccagcagcaggtgccccaaggaggccgtggtgttcgtgaccaagctgaagagggaggtgtgcgccgaccccaagaaggagnnngtgcagacctacatcaagaacctggacaggaaccagatgagg

SEQ ID NO: 31 - DNA, 234 bp, Mus musculus
  misc_feature 1..234: note = Description of sequence: Mouse CCL2 Y18X and Y33X
  source 1..234: mol_type = genomic DNA
  variation 52..54: note = N represents any nucleotide
  variation 97..99: note = N represents any nucleotide
ggcgccatgggcagccagcccgacgccgtgaacgcccccctgacctgctgcnnnagcttcaccagcaagatgatccccatgagcaggctggagagcnnnaagaggatcaccagcagcaggtgccccaaggaggccgtggtgttcgtgaccaagctgaagagggaggtgtgcgccgaccccaagaaggagtgggtgcagacctacatcaagaacctggacaggaaccagatgagg

SEQ ID NO: 32 - DNA, 234 bp, Mus musculus
  misc_feature 1..234: note = Description of sequence: Mouse CCL2 Y18X and W64X
  source 1..234: mol_type = genomic DNA
  variation 52..54: note = N represents any nucleotide
  variation 190..192: note = N represents any nucleotide
ggcgccatgggcagccagcccgacgccgtgaacgcccccctgacctgctgcnnnagcttcaccagcaagatgatccccatgagcaggctggagagctacaagaggatcaccagcagcaggtgccccaaggaggccgtggtgttcgtgaccaagctgaagagggaggtgtgcgccgaccccaagaaggagnnngtgcagacctacatcaagaacctggacaggaaccagatgagg

SEQ ID NO: 33 - DNA, 234 bp, Mus musculus
  misc_feature 1..234: note = Description of sequence: Mouse CCL2 Y33X and W64X
  source 1..234: mol_type = genomic DNA
  variation 97..99: note = N represents any nucleotide
  variation 97..99: note = N represents any nucleotide
ggcgccatgggcagccagcccgacgccgtgaacgcccccctgacctgctgctacagcttcaccagcaagatgatccccatgagcaggctggagagcnnnaagaggatcaccagcagcaggtgccccaaggaggccgtggtgttcgtgaccaagctgaagagggaggtgtgcgccgaccccaagaaggagnnngtgcagacctacatcaagaacctggacaggaaccagatgagg

SEQ ID NO: 34 - DNA, 234 bp, Mus musculus
  misc_feature 1..234: note = Description of sequence: Mouse CCL2 Y18X Y33X and W64X
  source 1..234: mol_type = genomic DNA
  variation 52..54: note = N represents any nucleotide
  variation 97..99: note = N represents any nucleotide
  variation 190..192: note = N represents any nucleotide
ggcgccatgggcagccagcccgacgccgtgaacgcccccctgacctgctgcnnnagcttcaccagcaagatgatccccatgagcaggctggagagcnnnaagaggatcaccagcagcaggtgccccaaggaggccgtggtgttcgtgaccaagctgaagagggaggtgtgcgccgaccccaagaaggagnnngtgcagacctacatcaagaacctggacaggaaccagatgagg

SEQ ID NO: 35 - protein, 99 aa, Homo sapiens
  REGION 1..99: note = Description of sequence: Full length Human WT CCL2
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 36 - protein, 148 aa, Mus musculus
  REGION 1..148: note = Description of sequence: Full length Mouse WT CCL2
MQVPVMLLGLLFTVAGWSIHVLAQPDAVNAPLTCCYSFTSKMIPMSRLESYKRITSSRCPKEAVVFVTKLKREVCADPKKEWVQTYIKNLDRNQMRSEPTTLFKTASALRSSAPLNVKLTRKSEANASTTFSTTTSSTSVGVTSVTVN